(19) United States
(12) Patent Application Publication
(10) Pub. No.: US 2006/0064170 A1
(43) Pub. Date: Mar. 23, 2006

(54) Closed system artificial intervertebral disc

(76) Inventors: Jeffrey A. Smith, Petaluma, CA (US); Michael S. Williams, Santa Rosa, CA (US)

Correspondence Address: DEANNA J. SHIRLEY, 3418 BALDWIN WAY, SANTA ROSA, CA, 95403, US

(21) Appl. No.: 11/216,581

(22) Filed: Aug. 30, 2005

Related U.S. Application Data
(60) Provisional application No. 60/611,161, filed on Sep. 17, 2004.

(51) Int. Cl. A61F 2/44

(52) U.S. Cl. 623/17.12; 623/17.16

(57) ABSTRACT

Devices and methods for manufacturing devices for treating degenerated and/or traumatized intervertebral discs are disclosed. Artificial discs and components of discs may include an artificial nucleus and/or an artificial annulus and may be comprised of shape memory materials synthesized to achieve desired mechanical and physical properties. An artificial nucleus and/or annulus according to the invention may comprise a first and second reservoir and one or more fluids, wherein upon application of a load upon the artificial nucleus and/or disc, the one or more fluids enters said second reservoir from said first reservoir.

### RELATED APPLICATIONS

This application is related U.S. Application Ser. No. 60/611,161 titled “Closed System Artificial Intervertebral Disc”, by Smith, et al., filed Sep. 17, 2004, the entirety of which is hereby incorporated as if fully set forth herein.

### FIELD OF THE INVENTION

The invention herein relates generally to medical devices and methods of treatment, and more particularly to devices and methods used in the treatment of a degenerated or traumatized intervertebral disc.

### BACKGROUND OF THE INVENTION

Intervertebral disc degeneration is a leading cause of pain and disability, occurring in a substantial majority of people at some point during adulthood. The intervertebral disc, comprising primarily the nucleus pulposus and surrounding annulus fibrosus, constitutes a vital component of the functional spinal unit. The intervertebral disc maintains space between adjacent vertebral bodies, absorbs impact between and cushions the vertebral bodies. The disc allows for fluid movement between the vertebral bodies, both subtle (for example, with each breath inhaled and exhaled) and dramatic (including rotational movement and bending movement in all planes.) Deterioration of the biological and mechanical integrity of an intervertebral disc as a result of disease and/or aging may limit mobility and produce pain, either directly or indirectly as a result of disruption of the functioning of the spine. Estimated health care costs of treating disc degeneration in the United States exceed $60 billion annually.

Age-related disc changes are progressive, and, once significant, increase the risk of related disorders of the spine. The degenerative process alters intradiscal pressures, causing a relative shift of axial load-bearing to the peripheral regions of the endplates and facets of the vertebral bodies. Such a shift promotes abnormal loading of adjacent intervertebral discs and vertebral bodies, altering spinal balance, shifting the axis of rotation of the vertebral bodies, and increasing risk of injury to these units of the spine. Further, the transfer of biomechanical loads appears to be associated with the development of other disorders, including both facet and ligament hypertrophy, osteophyte formation, lyphosis, spondylolisthesis, nerve damage, and pain.

In addition to age-related changes, numerous individuals suffer trauma-induced damage to the spine including the intervertebral discs. Trauma induced damage may include ruptures, tears, prolapse, herniations, and other injuries that cause pain and reduce strength and function.

Non-operative therapeutic options for individuals with neck and back pain include rest, analgesics, physical therapy, heat, and manipulation. These treatments fail in a significant number of patients. Current surgical options for spinal disease include discectomy, discectomy combined with fusion, and fusion alone. Numerous discectomies are performed annually in the United States. The procedure is effective in promptly relieving significant radicular pain, but, in general, the return of pain increases proportionally with the length of time following surgery. In fact, the majority of patients experience significant back pain by ten years following lumbar discectomy.

An attempt to overcome some of the possible reasons for failure of discectomy, fusion has the potential to maintain normal disc space height, to eliminate spine segment instability, and eliminate pain by preventing motion across a destabilized or degenerated spinal segment.

However, although some positive results are possible, spinal fusion may have harmful consequences as well. Fusion involves joining portions of adjacent vertebrae to one another. Because motion is eliminated at the treated level, the biomechanics of adjacent levels are disrupted. Resulting pathological processes such as spinal stenosis, disc degeneration, osteophyte formation, and others may occur at levels adjacent to a fusion, and cause pain in many patients. In addition, depending upon the device or devices and techniques used, surgery may be invasive and require a lengthy recovery period.

Consequently, there is a need in the art to treat degenerative disc disease and/or traumatized intervertebral discs, while eliminating the shortcomings of the prior art. There remains a need in the art to achieve the benefit of removal of a non-functioning intervertebral disc, to replace all or a portion of the disc with a device that will function as a healthy disc, eliminating pain, while preserving motion. There remains a need for an artificial disc or other device that maintains the proper intervertebral spacing, allows for motion, distributes axial load appropriately, and provides stability. In addition, an artificial disc requires secure long-term fixation to bone.

Further, there remains a need for an artificial nucleus that can be implanted within the annulus fibrosus, in order to restore normal disc functioning. Such a nucleus must comprise the characteristic lower durometer than the annulus fibrosus, must mimic the behavior of a healthy native nucleus upon load increase and decrease, and the annulus fibrosus must comprise the requisite stiffness as compared with the nucleus. Further, there remains a need for an artificial disc that can withstand typical cyclic stresses and perform throughout the life a patient. An artificial disc that can be implanted using minimally invasive techniques is also needed. And finally, a device that is compatible with current imaging modalities, such as Magnetic Resonance Imaging (MRI) is needed.

### SUMMARY OF THE INVENTION

An artificial nucleus and/or disc is disclosed comprising a substantially impermeable membrane, a first reservoir and a second reservoir within said first reservoir, wherein said second reservoir is substantially enclosed by a selectively permeable membrane, said first reservoir comprises one or more fluids, and wherein upon the application of a load to said artificial nucleus, some or all of said one or more fluids enters said second reservoir. Upon removal of said load, said one or more fluids may return to said first reservoir. The second reservoir of the artificial nucleus may comprise a plurality of substantially enclosed structures which may include a plurality of microspheres.

The first reservoir of the artificial nucleus may comprise a hydrogel. The artificial nucleus may comprise an elastic membrane.

### BRIEF DESCRIPTION OF THE DRAWINGS

FIG. 1 is a side view of a demonstrational system illustrating the principles of the invention, the system in a pre-load configuration.

FIG. 2 is a side view of the demonstrational system in a load configuration (following the application of a load).

FIG. 3 is a side view of the demonstrational system in a post-load configuration (following removal of the load imposed as illustrated above in FIG. 2.)

FIG. 4 is a cross-sectional view of an alternative closed system balloon according to the invention in a pre-load configuration.

FIG. 5 is a cross-sectional view of the closed system balloon of FIG. 4 following the application of a load.

FIG. 6 is a cross-sectional view of the closed system balloon following the removal of the load applied in FIG. 5.

FIG. 7 is a cross-sectional view of an artificial disc nucleus according to the invention in a pre-load configuration.

FIG. 8 is a cross-sectional view of the artificial disc nucleus of FIG. 7 in a load configuration (following the application of a load).

FIG. 9 is a cross-sectional view of the artificial disc nucleus of FIGS. 7 and 8 following the removal of a load.

FIG. 10 is a cross-section of an alternative closed system balloon according to the invention.

### DETAILED DESCRIPTION OF THE INVENTION

An endoprosthesis known as an artificial disc and/or an artificial disc nucleus are designed to replace a degenerated intervertebral disc. Such an artificial disc or disc nucleus may be expandable and/or self-expanding.

An “expandable” endoprosthesis comprises a reduced profile configuration and an expanded profile configuration. An expandable endoprosthesis according to the invention may undergo a transition from a reduced configuration to an expanded profile configuration via any suitable means, or may be self-expanding. Some embodiments according to the invention may comprise a substantially hollow interior that may be filled with a suitable medium, examples of which are set forth below. Such embodiments may accordingly be introduced into the body in a collapsed configuration, and, following introduction, may be filled to form a deployed configuration. Embodiments according to the invention may accordingly be implanted percutaneously or surgically. If implanted surgically, embodiments according to the invention may be implanted from either an anterior or a posterior approach, following the removal of some or all of the native disc, excepting the periphery of the native nucleus.

“Spinal fusion” is a process by which one or more adjacent vertebral bodies are adjoined to one another in order to eliminate motion across an unstable or degenerated spinal segment.

“Preservation of mobility” refers to the desired maintenance of normal motion between separate spinal segments.

“Spinal unit” refers to a set of the vital functional parts of the spine including a vertebral body, endplates, facets, and intervertebral disc.

The term “cable” refers to any generally elongate member fabricated from any suitable material, whether polymeric, metal or metal alloy, natural or synthetic.

The term “fiber” refers to any generally elongate member fabricated from any suitable material, whether polymeric, metal or metal alloy, natural or synthetic.

As used herein, the term “braid” refers to any braid or mesh or similar wound or woven structure produced from between 1 and several hundred longitudinal and/or transverse elongate elements wound, woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, radiation, including but not limited to radiation in the ultraviolet range, and others. Some embodiments according to the invention may comprise one or more polymers having a structure that assumes a first configuration, a second configuration, and a hydrophilic polymer of sufficient rigidity coated upon at least a portion of the structure when the device is in the second configuration. Upon placement of the device in an aqueous environment and consequent hydration of the hydrophilic polymer, the polymer structure reverts to the first configuration.

Some embodiments according to the invention, while not technically comprising shape memory characteristics, may nonetheless readily convert from a constrained configuration to a deployed configuration upon removal of constraints, as a result of a material's elasticity, super-elasticity, a particular method of “rolling down” and constraining the device for delivery, or a combination of the foregoing. Such embodiments may comprise one or more elastomeric or rubber materials.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions.

“Transition temperature” refers to the temperature above which a shape memory polymer reverts to its original memorized configuration.

The term “strain fixity rate” R_{f} is a quantification of the fixability of a shape memory polymer's temporary form, and is determined using both strain and thermal programs. The strain fixity rate is determined by gathering data from heating a sample above its melting point, expanding the sample to 200% of its temporary size, cooling it in the expanded state, and drawing back the extension to 0%, and employing the mathematical formula:
R_{f}(N)=εᵤ(N)/εₘ
where εᵤ(N) is the extension in the tension-free state while drawing back the extension, and εₘ is 200%.
The “strain recovery rate” Rᵣ describes the extent to which the permanent shape is recovered:

$${R}_{r} ( N ) = \frac{{ɛ}_{m} - {ɛ}_{p} ( N )}{{ɛ}_{m} - {ɛ}_{p} ( N - 1 )}$$

where εₚ is the extenstion at the tension free state.

A “switching segment” comprises a transition temperature and is responsible for the shape memory polymer's ability to fix a temporary shape.

A “thermoplastic elastomer” is a shape memory polymer comprising crosslinks that are predominantly physical crosslinks.

A “thermoset” is a shape memory polymer comprising a large number of crosslinks that are covalent bonds.

Shape memory polymers are highly versatile, and many of the advantageous properties listed above are readily controlled and modified through a variety of techniques. Several macroscopic properties such as transition temperature and mechanical properties can be varied in a wide range by only small changes in their chemical structure and composition. More specific examples are set forth in Provisional U.S. Patent Application Ser. No. 60/523,578 and are incorporated in their entirety as if fully set forth herein.

Shape memory polymers are characterized by two features, triggering segments having a thermal transition Tₜᵣₐₙₛ within the temperature range of interest, and crosslinks determining the permanent shape. Depending on the kind of crosslinks (physical versus covalent bonds), shape memory polymers can be thermoplastic elastomers or thermosets. By manipulating the types of crosslinks, the transition temperature, and other characteristics, shape memory polymers can be tailored for specific clinical applications.

More specifically, according the invention herein, one can the control shape memory behavior and mechanical properties of a shape memory polymer through selection of segments chosen for their transition temperature, and mechanical properties can be influenced by the content of respective segments. The extent of crosslinking can be controlled depending on the type of material desired through selection of materials where greater crosslinking makes for a tougher material than a polymer network. In addition, the molecular weight of a macromonomeric crosslinker is one parameter on the molecular level to adjust crystallinity and mechanical properties of the polymer networks. An additional monomer may be introduced to represent a second parameter.

Further, the annealing process (comprising heating of the materials according to chosen parameters including but not limited to time and temperature) increases polymer chain crystallization, thereby increasing the strength of the material. Consequently, according to the invention, the desired material properties can be achieved by using the appropriate ratio of materials and by annealing the materials.

Additionally, the properties of polymers can be enhanced and differentiated by controlling the degree to which the material crystallizes through strain-induced crystallization. Means for imparting strain-induced crystallization are enhanced during deployment of an endoprosthesis according to the invention. Upon expansion of an endoprosthesis according to the invention, focal regions of plastic deformation undergo strain-induced crystallization, further enhancing the desired mechanical properties of the device, such as further increasing radial strength. The strength is optimized when the endoprosthesis is induced to bend preferentially at desired points.

Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(.beta.-hydroxybutyrate), poly(3-hydroxyoctanoate) and poly(3-hydroxyfatty acids).

Suitable synthetic polymer blocks include polyphosphazenes, poly(vinyl alcohols), polyamides, polyester amides, poly(amino acid)s, synthetic poly(amino acids), polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, polyethylene terephthalate, polysiloxanes, polyurethanes, fluoropolymers (including but not limited to polyfluorotetraethylene), and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

For those embodiments comprising a shape memory polymer, the degree of crystallinity of the polymer or polymeric block(s) is between 3 and 80%, more often between 3 and 65%. The tensile modulus of the polymers below the transition temperature is typically between 50 MPa and 2 GPa (gigapascals), whereas the tensile modulus of the polymers above the transition temperature is typically between 1 and 500 MPa. Most often, the ratio of elastic modulus above and below the transition temperature is 20 or more.

The melting point and glass transition temperature of the hard segment are generally at least 10 degrees C., and preferably 20 degrees C., higher than the transition temperature of the soft segment. The transition temperature of the hard segment is preferably between −60 and 270 degrees C., and more often between 30 and 150 degrees C. The ratio by weight of the hard segment to soft segments is between about 5:95 and 95:5, and most often between 20:80 and 80:20. The shape memory polymers contain at least one physical crosslink (physical interaction of the hard segment) or contain covalent crosslinks instead of a hard segment. The shape memory polymers can also be interpenetrating networks or semi-interpenetrating networks. A typical shape memory polymer is a block copolymer.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

Examples of highly elastic materials including but not limited to vulcanized rubber, polyurethanes, thermoplastic elastomers, and others may be used according to the invention.

Curable materials include any material capable of being able to transform from a fluent or soft material to a harder material, by cross-linking, polymerization, or other suitable process. Materials may be cured over time, thermally, chemically, or by exposure to radiation. For those materials that are cured by exposure to radiation, many types of radiation may be used, depending upon the material. Wavelengths in the spectral range of about 100-1300 nm may be used. The material should absorb light within a wavelength range that is not readily absorbed by tissue, blood elements, physiological fluids, or water. Ultraviolet radiation having a wavelength ranging from about 100-400 nm may be used, as well as visible, infrared and thermal radiation. The following materials are some examples of curable materials: urethanes, polyurethane oligomer mixtures, acrylate monomers, aliphatic urethane acrylate oligomers, acrylamides, UV curable epoxies, photopolymerizable polyanhydrides and other UV curable monomers. Alternatively, the curable material can be a material capable of being chemically cured, such as silicone based compounds which undergo room temperature vulcanization.

Though not limited thereto, some embodiments according to the invention comprise one or more therapeutic substances that will elute from the surface. Suitable therapeutics include but are not limited to bone growth accelerators, bone growth inducing factors, osteoinductive agents, immunosuppressive agents, steroids, anti-inflammatory agents, pain management agents (e.g, analgesics), tissue proliferative agents to enhance regrowth and/or strengthening of native disc materials, and others. According to the invention, such surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”).

The use of polymeric materials in the fabrication of endoprostheses confers the advantages of improved flexibility, compliance and conformability. Fabrication of an endoprosthesis according to the invention allows for the use of different materials in different regions of the prosthesis to achieve different physical properties as desired for a selected region. An endoprosthesis comprising polymeric materials has the additional advantage of compatibility with magnetic resonance imaging, potentially a long-term clinical benefit.

As set forth above, some embodiments according to the invention may comprise components that have a substantially hollow interior that may be filled after being delivered to a treatment site with a suitable material in order to place the device in a deployed configuration. Accordingly, such embodiments may comprise a fluid retention bag having a membrane layer comprising polyvinyl chloride (PVC), polyurethane, and or laminates of polyethylene terephthalate (PET) or nylon fibers or films within layers of PVC, polyurethane or other suitable material. Such a fluid retention bag or membrane layer alternatively may comprise Kevlar, polyimide, a suitable metal, or other suitable material within layers of PVC, polyurethane or other suitable material. Such laminates may be of solid core, braided, woven, wound, or other fiber mesh structure, and provide stability, strength, and a controlled degree of compliance. Such a laminate membrane layer may be manufactured using radiofrequency or ultrasonic welding, adhesives including ultraviolet curable adhesives, or thermal energy.

A fluid retention bag as set forth above may be filled with any suitable material including but not limited to saline, contrast media, hydrogels, a polymeric foam, or any combination thereof. A polymeric foam may comprise a polyurethane intermediate comprising polymeric diisocyanate, polyols, and a hydrocarbon, or a carbon dioxide gas mixture. Such a foam may be loaded with any of numerous solid or liquid materials known in the art that confer radiopacity.

Such a fluid retention membrane and/or bag may be designed to replace an entire intervertebral disc. Alternatively, it may replace only the nucleus pulposus or only the annulus fibrosus. Such a device may comprise one or more filling ports, and include separate filling ports for the nucleus pulposus and annulus fibrosus, to allow for varying durometers, and possibly varied materials in order to mimic the properties of the native disc components.

Such a device may comprise a single unit, or may be two or more individual parts. If the device comprises two or more component parts, the parts may fit together in a puzzle-like fashion. The device may further comprise alignment tabs for stable alignment between the vertebral bodies.

Such a fluid retention membrane and/or bag may comprise interbody connections and/or baffles and/or partitions or generally vertically oriented membranes in order to maintain structural integrity after filling, to increase the devices ability to withstand compressive, shear, and other loading forces, and/or to direct filling material flow and positioning, and/or to partition portions of the disc in order to separate injection of different types or amounts of filling materials.

Following surgical or minimally invasive surgical access and removal of all or a portion of the native disc, a deflated fluid retention bag or membrane may be delivered to the intervertebral space surgically or through a catheter and/or cannula. The membrane and/or bag is positioned within the intervertebral space. The membrane inflation port or ports are then attached to the injection source. Filling material is then injected. Following injection of the filling material, which may be curable by any suitable means or may be catalytically activated or may remain in fluid form, the injection source is detached and removed.

Details of the invention can be better understood from the following descriptions of specific embodiments according to the invention which are set forth as examples of the general principles of the invention. It will be appreciated that numerous structural and material modifications may be made without departing from the spirit and scope of the invention. It will also be appreciated that the following embodiments may serve as an artificial disc nucleus, artificial disc annulus, or both. FIG. 1 illustrates the principles of the invention herein via a cross section of system 5. System 5 comprises cylindrical chamber 10, piston 15, hydrogel 20 and second reservoir 25. Shown in cross section in FIG. 1, piston 15 is in a first configuration and with chamber 10 defines first reservoir 12. Second reservoir 25 comprises permeable membrane 28, but alternative embodiments according to the invention may comprise an impermeable membrane. In the pre-load configuration illustrated in FIG. 1, system 5 is at equilibrium.

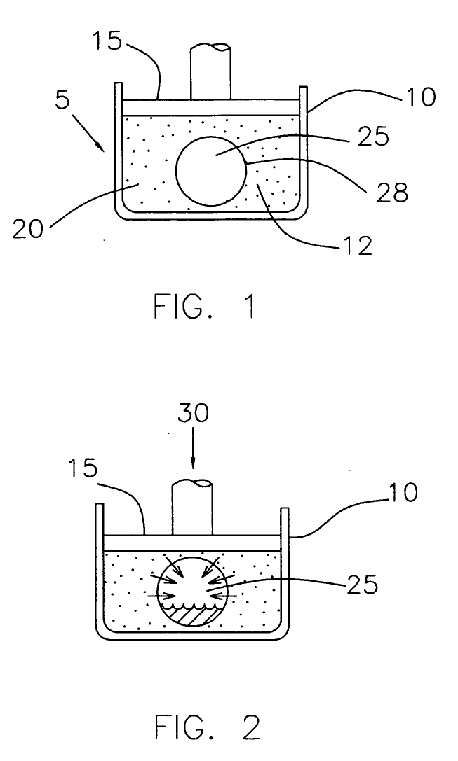

In FIG. 2, load 30 is applied to piston 15, exerting a downward force on piston 50. As a result of the increased pressure place upon hydrogel 20, water from within hydrogel 20 is forced through permeable membrane 28 into interior 27 of second reservoir 25. Consequently, the volume of first reservoir 12 decreases. Hydrogel 20 then comprises a lower volume of water. The extent to which water is forced into the interior of second reservoir 25 depends upon the magnitude of load 30.

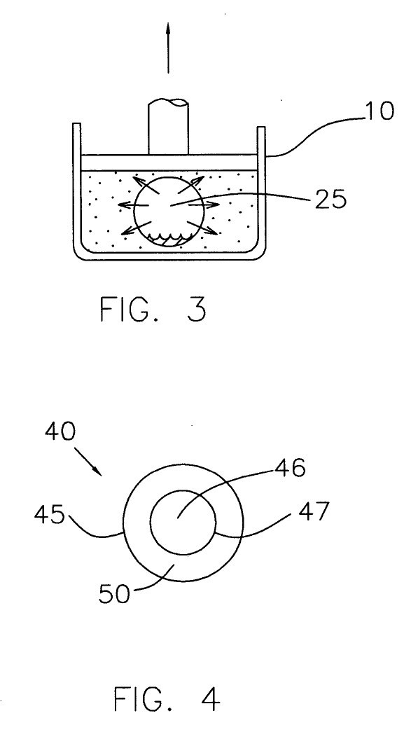

FIG. 3 illustrates the interactive behavior of second reservoir 25 following the removal of load 30. With the decrease in pressure on system 5, previously dehydrated hydrogel 20 “pulls” water from the interior of second reservoir 25 through permeable membrane 28. Hydrogel 20 is progressively rehydrated, and interior 27 of second reservoir 25 empties partially or completely. Upon reapplication of a force, the foregoing system 5 will again undergo the foregoing relational and configurational steps. System 5 represents the repeated load bearing and unloading of a functional spine, and the behavior of an artificial disc according to the invention during the repeated application and removal of a load.

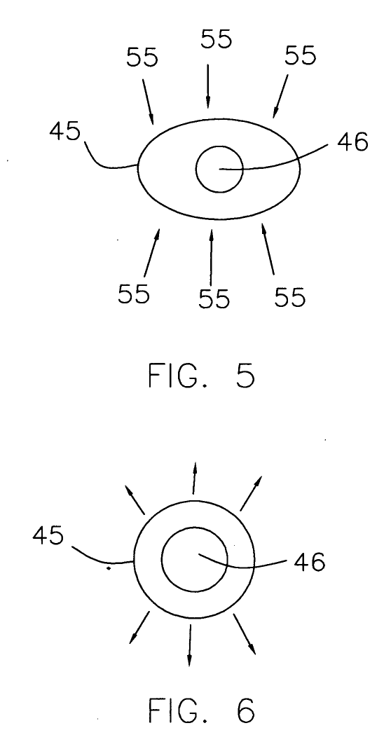

Turning now to FIG. 4, an alternative embodiment of the invention is illustrated in a preload configuration. Artificial nucleus 40 comprises impermeable elastic bag 45, impermeable reservoir 47 and hydrogel 50. Upon application of multidirectional load 55, as illustrated in FIG. 5, pressure is transferred to impermeable reservoir 47 and decreases volume of impermeable reservoir 47. Consequently, the volume of elastic bag 45 decreases. The extent to which the volume of impermeable reservoir decreases depends upon the magnitude of the load applied.

Upon removal of load 55, as illustrated in FIG. 6, pressure upon gas 46 decreases. Consequently, the volume of impermeable reservoir 47 increases to its original pre-load volume. Similar to the embodiment discussed in relation to FIGS. 1-3, artificial nucleus 40 can undergo numerous repetitions of the foregoing cycle.

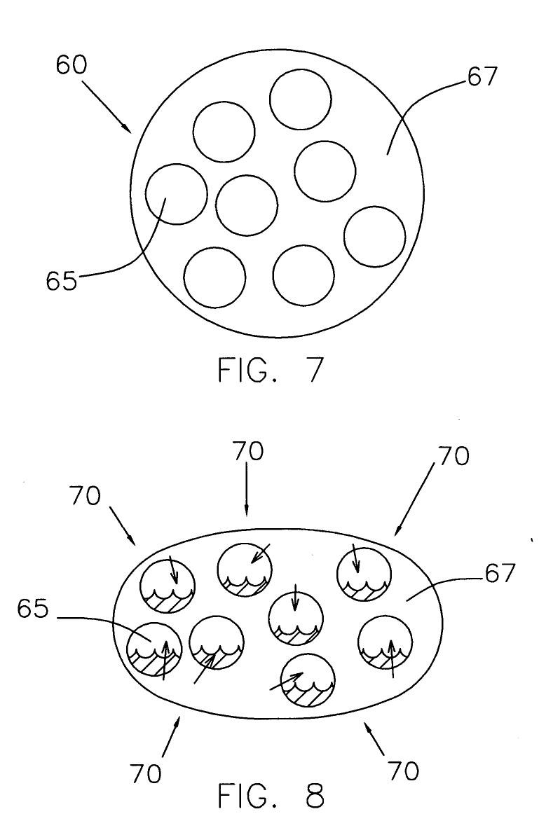

FIGS. 7-9 illustrate artificial disc 60 in cross section. Artificial disc 60 comprises microsphere reservoir 65 and hydrogel 67. Upon application of multidirectional load 70, as illustrated in FIG. 8, pressure forces water from hydrogel 67 into the interior of microsphere reservoir 65. Consequently, the volume of artificial disc 60 decreases. The extent to which water is forced into the interior of microsphere reservoir 65 depends upon the magnitude of the load applied.

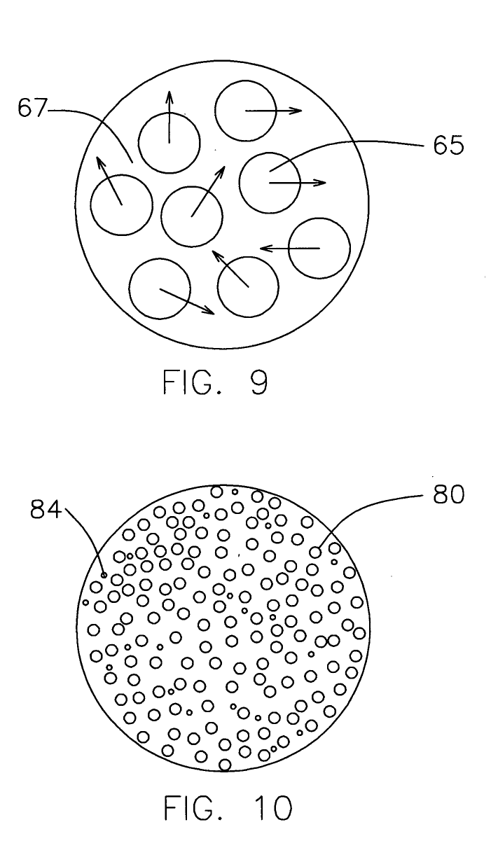

Upon removal of load 70, as illustrated in FIG. 9, dehydrated hydrogel 67 “pulls” water from the interior of microsphere reservoir 65, and is thereby rehydrated. Similar to the embodiment discussed in relation to FIGS. 1-6, artificial disc 60 can undergo numerous repetitions of the foregoing cycle.

FIG. 10 illustrates an embodiment similar to that discussed above in relation to FIGS. 7-9. However, the example of FIG. 10 illustrates a greater concentration of microspheres 80 within hydrogel matrix 84 of microsphere reservoir 85 than the example of FIGS. 7-9.

Desirable materials for use in the manufacture of elastic bags and/or impermeable reservoirs include, by way of example, polymers, elastomeric, viscoelastic, super elastic polymers and shape memory polymers.

While all of the foregoing embodiments can most advantageously be delivered in a minimally invasive, percutaneous manner, the foregoing embodiments may also be implanted surgically. Further, while particular forms of the invention have been illustrated and described above, the foregoing descriptions are intended as examples, and to one skilled in the art it will be apparent that various modifications can be made without departing from the spirit and scope of the invention.

We claim:

1. An artificial nucleus comprising a substantially impermeable membrane, a first reservoir and a second reservoir within said first reservoir, wherein said second reservoir is substantially enclosed by a selectively permeable membrane, said first reservoir comprises one or more fluids, and wherein upon the application of a load to said artificial nucleus, some or all of said one or more fluids enters said second reservoir.

2. The artificial nucleus according to claim 1 wherein upon removal of said load, said one or more fluids returns to said first reservoir.

3. The artificial nucleus according to claim 1 wherein said second reservoir comprises a plurality of substantially enclosed structures.

4. The artificial nucleus according to claim 3 wherein said second reservoir comprises a plurality of microspheres.

5. The artificial nucleus according to claim 1 wherein said first reservoir comprises a hydrogel.

6. The artificial nucleus according to claim 1 wherein said nucleus comprises an elastic membrane.

7. An artificial disc comprising a substantially impermeable membrane, a first reservoir and a second reservoir within said first reservoir, wherein said second reservoir is substantially enclosed by a selectively permeable membrane, said first reservoir comprises one or more fluids, and wherein upon the application of a load to said artificial disc, some or all of said one or more fluids enters said second reservoir.

8. The artificial disc according to claim 1 wherein upon removal of said load, said one or more fluids returns to said first reservoir.

9. The artificial disc according to claim 1 wherein said second reservoir comprises a plurality of substantially enclosed structures.

10. The artificial disc according to claim 3 wherein said second reservoir comprises a plurality of microspheres.

11. The artificial disc according to claim 1 wherein said first reservoir comprises a hydrogel.

12. The artificial disc according to claim 1 wherein said disc comprises an elastic membrane.
